(19) United States
(12) Patent Application Publication
(10) Pub. No.: US 2022/0291244 A1
(43) Pub. Date: Sep. 15, 2022

(54) KAWASAKI DISEASE DETERMINATION KIT AND KAWASAKI DISEASE DETERMINATION METHOD

(71) Applicants: FUKUOKA CITY HOSPITAL ORGANIZATION, Fukuoka-shi, Fukuoka (JP); PREMEDICA INC., Tokyo (JP)

(72) Inventors: Toshiro HARA, Fukuoka-shi, Fukuoka (JP); Hiroshi SAKAZUME, Tokyo (JP)

(73) Assignees: FUKUOKA CITY HOSPITAL ORGANIZATION, Fukuoka-shi, Fukuoka (JP); PREMEDICA INC., Tokyo (JP)

(21) Appl. No.: 17/638,307

(22) PCT Filed: Sep. 4, 2020
(86) PCT No.: PCT/JP2020/033516

§ 371 Date: Feb. 25, 2022

(30) Foreign Application Priority Data
Sep. 6, 2019 (JP) 2019-163111

(51) Int. Cl. G01N 33/92

(52) U.S. Cl. CPC G01N 33/92; G01N 2333/775; G01N 2800/328

(57) ABSTRACT

A biomarker for determining Kawasaki disease is identified by lipidomic analysis and mass spectrometry. With the use of the biomarker, we develop and provide a kit and a method capable of directly and objectively determining whether the subject suspected of having Kawasaki disease suffers from Kawasaki disease. A kit for determining Kawasaki disease, the kit including LOX-1 protein and/or part thereof having LAB-binding ability, the protein or part thereof being immobilized on a surface of a base material, is provided.

### TECHNICAL FIELD

The present invention relates to a kit for determining Kawasaki disease and a method for determining Kawasaki disease.

### BACKGROUND ART

Kawasaki disease (hereinafter, often referred to as “KD”) is a febrile disease that characteristically exhibits systemic vasculitis. KD occurs mainly in childhood under the age of 5 years. When the disease is not treated, about 25 to 30% of KD patients is associated with coronary artery lesions (CAL), which may be a risk factor for development of myocardial infarction due to thrombogenesis or the like. Therefore, KD is now recognized as a main cause of acquired heart diseases in childhood in developed countries (Non-patent Literature 1 to 3).

More than half a century has now passed since the first report of KD in 1967. However, the cause of the disease and its association with adult cardiovascular diseases have not yet been clarified (Non-patent Literature 1 and 4).

Environmental epidemiological research by Toronto Children's Hospital has suggested that the children living in a hygienic environment with less external stimulations such as allergens and atmospheric biological particles may develop KD on exposure to a certain infectious or environmental triggering factor thereafter (Non-patent Literature 5). It is thus thought that, in KD, excessive immune response occurs due to a certain environmental factor, and causes vasculitis in children having a specific genetic factor.

Although a number of pathogens have been reported as causes of KD, in the most studies, the results obtained among different cohorts do not match. The only exception is Yersinia pseudotuberculosis belonging to Enterobacteriaceae. In Japan, about 10% of patients infected with Y. pseudotuberculosis have actually developed KD (Non-patent Literature 6). Also in Europe, an increase in the incidence of KD was found when infection with Y. pseudotuberculosis was prevalent. Moreover, it is known that KD patients infected with Y. pseudotuberculosis exhibit a higher incidence of coronary artery lesions (CAL) relative to uninfected patients (Non-patent Literature 7).

These results have demonstrated that the innate immune system plays an important role in the development of KD, although the mechanism of development and the causative factor of KD have not been clarified yet. Therefore, no objective method of examination for diagnosing KD has been established yet, and the diagnosis still depends on clinical findings, and exclusion diagnosis of other diseases based on the clinical findings. Thus, diagnosis of KD tends to suffer from subjectivity of the doctor or misdiagnosis, which is problematic.

### CITATION LIST

### Non-Patent Literature

Non-patent Literature 1: Hara T., et al., 2016, Clin Exp Immunol, 186: 134-143.

Non-patent Literature 2: McCrindle B. W., et al., 2017, Circulation, 135: e927-e999.

Non-patent Literature 3: Nakamura Y., 2018, Int J Rheum Dis, 21: 16-19.

Non-patent Literature 4: Burgner D. & Hamden A., 2005, Int J Infect Dis, 9: 185-194.

Non-patent Literature 5: Manlhiot C, et al., 2018, PLoS One, 13: e0191087.

Non-patent Literature 6: Sato K. et al., 1983, Pediatr Infect Dis, 2: 123-126.

Non-patent Literature 7: Vincent P., 2007, Pediatr Infect Dis J 26:629-63.

### SUMMARY OF INVENTION

### Technical Problem

An object of the present invention is to identify a marker for determining KD, and to use as a biomarker for developing and providing a kit and a method capable of directly and objectively determining whether the subject suspected of having KD suffers from KD.

### Solution to Problem

In order to solve the above problem, the present inventors carried out a lipidomics analysis by an LC-MS (liquid chromatography mass spectrometry) analysis method using sera of KD patient groups to find a causative factor of KD. As a result, a number of molecules could be identified as “Kawasaki disease-specific molecules”. Twenty-eight molecules among these molecules were found to be detected repeatedly among a plurality of different KD patient groups (unpublished data). Furthermore, two of the molecules were found to be associated with a coronary artery lesion known to be associate with KD. As a result of structural analysis of these two molecules using an LC-MS/MS (LC-tandem mass spectrometry) analysis method, one molecule was found to have an oxidized phosphatidylcholine (oxidized PC) structure. Oxidized PC becomes LAB (Lox-1 ligand containing apolipoprotein B) by binding to apolipoprotein B. LAB is known to be a molecule associated with development of arteriosclerosis, and to specifically bind to LOX-1 protein (lectin-like oxidized low-density lipoprotein receptor 1 protein). However, its association with KD has not been reported. In view of this, the amounts of LAB in plasmas of KD patients and healthy individuals were examined using LOX-1 protein as a capturing material. As a result, KD patients were found to exhibit significantly higher amounts of LAB. In the convalescent phase, the plasma levels of LAB in the KD patients decreased to show no significant difference from those in the healthy individuals. These results suggest that LAB may potentially be a biomarker for KD. The present invention is based on the above novel discovery on KD, and provides the following.

(1) A kit for determining Kawasaki disease, the kit comprising a LAB-capturing device,

the LAB-capturing device comprising lectin-like oxidized low-density lipoprotein receptor 1 protein (LOX-1 protein) and/or part thereof having LAB-binding ability, the protein and/or part thereof being immobilized on a surface of a base material.

(2) The kit for determining Kawasaki disease according to (1), wherein the LOX-1 protein is any of the polypeptides shown in the following (a) to (c):

(a) a polypeptide having the amino acid sequence represented by SEQ ID NO:2;

(b) a polypeptide derived from the amino acid sequence represented by SEQ ID NO:2 by deletions, substitutions or additions of one or more amino acids; and

(c) a polypeptide having 90% or more an amino acid identity to the amino acid sequence represented by SEQ ID NO:2.

(3) The kit for determining Kawasaki disease according to (1), wherein the part is any of the polypeptides shown in the following (d) to (0:

(d) a polypeptide having the amino acid sequence represented by any of SEQ ID NOs:3 to 5;

(e) a polypeptide derived from the amino acid sequence represented by any of SEQ ID NOs:3 to 5 by deletions, substitutions or additions of one or more amino acids; and

(f) a polypeptide having 90% or more an amino acid identity to the amino acid sequence represented by any of SEQ ID NOs:3 to 5.

(4) The kit for determining Kawasaki disease according to any one of (1) to (3), further comprising a LAB-detecting agent.

(5) The kit for determining Kawasaki disease according to (4), wherein the LAB-detecting agent is labeled.

(6) The kit for determining Kawasaki disease according to (4) or (5), wherein the LAB-detecting agent is an anti-LAB antibody or a fragment thereof having LAB-binding ability.

(7) A method for determining Kawasaki disease, the method comprising:

a measurement step of measuring the amount of LAB contained per unit amount of blood sample collected from a subject to obtain a measured value of the amount of LAB; and

a determination step of determining whether the subject suffers from Kawasaki disease based on the measured value obtained in the measurement step.

(8) The method for determining Kawasaki disease according to (7), wherein, in the determination step, the subject is determined to have Kawasaki disease, when the measured value obtained in the measurement step is higher than a predetermined cut-off value, or when the measured value obtained in the measurement step is significantly higher than the amount of LAB contained per unit amount of blood sample collected from a healthy-individual group.

(9) The method for determining Kawasaki disease according to (7) or (8), wherein the measurement in the measurement step is carried out using receptor-ligand activity between LAB, and LOX-1 protein and/or a part thereof having LAB-binding ability.

(10) The method for determining Kawasaki disease according to any one of (7) to (9), wherein the blood sample is any of blood, serum, and plasma.

(11) Use of LAB as a biomarker to be used for determining Kawasaki disease.

The present description includes the disclosure of Japanese Patent Application Number 2019-163111 as the basis of the priority of the present application.

### Advantageous Effects of Invention

According to the present invention, by application of a method for determining KD using a marker for determining whether the subject suspected of having KD suffers from KD, diagnosis of KD, which had been inevitably dependent on clinical findings and exclusion diagnosis, can be directly and objectively judged.

### BRIEF DESCRIPTION OF DRAWING

FIG. 1 is a box plot illustrating the LAB levels in the plasma of KD patients. In the figure, “Control” represents plasma samples from normal control individuals (n=5), and “Disease Control” represents plasma samples from disease control (n=7). Regarding Kawasaki disease (KD), “Acute” represents plasma samples derived from KD patients in the acute phase (n=16), and “Convalescent” represents plasma samples from patients in the convalescent phase who had previously had KD (n=8). In the figure, “*” represents p<0.05, and “***” represents p<0.001 (Tukey's HSD test).

### DESCRIPTION OF EMBODIMENTS

### 1. Kit for Determining Kawasaki Disease (Kit for Determining KD)

### 1-1. Summary

A first aspect of the present invention is a kit for determining Kawasaki disease (kit for determining KD). The kit of the present invention comprises a LAB-capturing device as an essential component, and a LAB-detecting agent as a selective component. According to the kit for determining KD of the present invention, KD, whose diagnostic method has conventionally been limited to exclusion diagnosis based on clinical findings, can be highly accurately and sensitively determined, so that the kit can assist the diagnosis of KD by medical doctors.

### 1-2. Definitions

Terms frequently used in the present description are defined as follows. As described above, “Kawasaki disease (1KD)” is a childhood febrile disease that characteristically exhibits systemic vasculitis. In the present description, the disease is the target disease to determine whether the subject suffers from KD.

“Determining Kawasaki disease (KD)” means determination of whether a subject suffers from KD, that is, a diagnostic aid for determination of whether a subject is suspected of having KD.

“LOX-1 (lectin-like oxidized low-density lipoprotein receptor-1/lectin-like oxidized LDL receptor-1) protein” (often referred to as “LOX-1 protein” in the present description) is a single-transmembrane receptor protein whose N-terminus is intracellularly exposed, and whose C-terminus is extracellularly exposed. The protein forms a homodimer via disulfide bond, and is expressed in vascular endothelial cells, smooth muscles, macrophages, and the like. The protein functions as a scavenger receptor of oxidized LDL, as described later, and is recently known as a promoting factor of arteriosclerosis. Expression of LOX-1 protein is known to be induced by platelets, endothelial cells, vascular smooth muscle cells, ischemia-reperfusion injury in neurons and macrophages, active oxygen, and inflammatory cytokines.

“LDL (low-density lipoprotein)” means a lipoprotein having a low specific gravity and formed by binding of a protein component consisting of apo-protein B (apoB) to a lipid component consisting of cholesterol, triglyceride, and phospholipid. LDL has a function that transports cholesterol produced in the liver to the whole body via blood. Since an increase in the blood LDL level is a risk factor for arteriosclerosis, LDL is generally called “bad cholesterol”.

“Oxidized LDL (ox-LDL)” means LDL whose lipid component or protein component has undergone oxidative modification or oxidative damage due to free radicals such as active oxygen. Oxidized LDL is also called modified LDL or LAB (LOX-1 ligand containing apolipoprotein B). In the present description, unless otherwise specified, oxidized LDL or modified LDL is represented as “LAB”. Since KD patients exhibit significant increases in the blood level of LAB, LAB is used as a marker for determining KD in the present description.

In present description, the “marker for determining KD” is a biomarker consisting of LAB capable of determining whether the subject suffers from KD.

### 1-3. Configuration

The kit for determining KD of the present description comprises a LAB-capturing device as an essential component, and a LAB-detecting agent as a selective component. Each component is specifically described below.

### 1-3-1. LAB-Capturing Device

In present description, the “LAB-capturing device” comprises a base material, and LOX-1 protein and/or a part thereof immobilized on a surface thereof

### (1) Base Material

The “base material” in the present invention is a solid-phase carrier for immobilizing LOX-1 protein and/or a part thereof.

The quality of the base material is not limited as long as LOX-1 protein and/or the part thereof can at least be directly or indirectly immobilized on the surface thereof. The quality of the base material is preferably, but does not necessarily need to be, a water-insoluble material. Examples of the quality include plastics; glasses; metals; ceramics; natural resins (such as natural rubber and Japanese lacquer); natural and chemical fibers, and combined materials thereof (such as papers, unwoven fabrics, and filters); polysaccharide polymers such as agar; gelled proteins (such as gelatin and collagen); and mixtures thereof. The quality of the base material may be appropriately selected depending on the measurement method for LAB. For example, in cases of measurement by an enzyme immunoassay method such as the ELISA method, fluorescence method, or colorimetric method, the quality of the base material is preferably, but does not necessarily need to be, a plastic or glass in light of the cost, processability, operability, and the like. Transparent materials are preferred. Specific examples of plastics that may be used include polyvinyl chloride, polyvinylidene chloride, polystyrene, polyurethane, polysulfone, polycarbonate, polyarylate, polyamide, and polyvinyl alcohol. In cases of measurement by an SPR measurement sensor, a QCM measurement sensor, or the like, it is preferred to use a metal forming the sensor chip, such as gold (Au), platinum (Pt), silver (Ag), or copper (Cu).

The shape of the base material may be appropriately decided depending on the use of the kit of the present invention. Examples of the shape include plates (including square plates such as 96-well microtiter plates), dishes, tubes, sticks, beads, plates, and test strips. In cases of formation on the surface of a bead, the base material may be a sphere having a diameter of about 1 μm to about 1 cm. Further, for example, in cases of using the kit of the present invention for a sensor chip of an SPR measurement sensor, the base material may be in a shape suitable for the SPR measurement sensor employed.

The base material may be a multilayer structure consisting of two or more materials. For example, a base material formed by laminating a metal film on a glass surface corresponds to the multilayer structure. In cases of the base material with such a multilayer structure, at least the layer forming the base material surface needs to be the quality of the base material on which LOX-1 protein and/or part thereof can be immobilized.

The “base material surface” means a base material moiety that can directly contact with the blood sample collected from the subject. Thus, the base material surface may vary depending on the shape of the base material employed. For example, when the base material has a plate-like shape such as the slide glass, the base material surface corresponds to the front side, back side, and/or lateral sides. When the base material has a tubular shape, the base material surface corresponds to the outer side, inner side, and cross-section of the tube. When the base material has a spherical shape, the base material surface generally corresponds to the outer surface of the sphere, and, when the base material has an inner space open to the outside, the base material surface also includes the inner surface. Examples of such cases include cases where the base material is a hollow bead or a porous material.

“Immobilization on a base material surface” means immobilization of a peptide on a base material surface. The peptide in present description corresponds especially to LOX-1 protein and/or part thereof. The method for the immobilization is not limited. Examples of the method include chemical adsorption, physical adsorption, and affinity. Examples of the chemical adsorption include chemical bonds such as covalent bonds and ionic bonds. The physical adsorption includes the van der Waals force.

### (2) LOX-1 Protein

The LOX-1 protein in present description is a receptor protein of LAB, which is a marker for determining the presence of KD in blood samples. Thus, LOX-1 protein has LAB-binding ability based on the receptor-ligand activity. In present description, “receptor-ligand activity” means a specific protein-protein binding affinity activity that occurs between a ligand and receptor thereof. Thus, in the LAB-capturing device, LOX-1 protein and the later-described the part thereof function as capturing materials that bind to LAB based on the receptor-ligand activity, to enable the detection.

Unless otherwise specified, “LOX-1 protein” in present description means human LOX-1 protein. The LOX-1 protein includes the wild type and mutant types. More specifically, the “wild-type LOX-1 protein” corresponds to human LOX-1 protein consisting of the amino acid sequence represented by SEQ ID NO:2. The “mutant-type LOX-1 protein” means a polypeptide which has a mutation(s) in part thereof, and keeps the LAB-binding ability. Examples of the mutant-type LOX-1 protein include: polypeptides derived from the amino acid sequence represented by SEQ ID NO:2by deletions, substitutions or additions of one or more amino acids ; and polypeptides having 90% or more, 91% or more, 92% or more, 93% or more, 94% or more, 95% or more, 96% or more, 97% or more, 98% or more, or 99% or more an amino acid identity to the amino acid sequence represented by SEQ ID NO:2. Specific examples of the mutant-type LOX-1 protein include, but are not limited to, splicing variants, and mutants based on SNIPs and/or the like.

In present description, “plurality” means, for example, 2 to 20, 2 to 15, 2 to 10, 2 to 7, 2 to 5, 2 to 4, or 2 to 3. The “amino acid identity” means the ratio (%) of matched amino acid residues in the total number of amino acid residues, which ratio is calculated by performing alignment of the amino acid sequences of two polypeptide to be compared such that the number of matched amino acid residues becomes maximum by, if necessary, inserting a gap(s) in one or both of the sequences. The alignment of the two amino acid sequences for the calculation of the amino acid identity may be carried out using a known program such as Blast, FASTA, or Clustal W.

The “(amino acid) substitution” in present description means substitution within a conservative group of amino acids having similar properties in terms of the charge, side chain, polarity, aromaticity, and/or the like among the 20 kinds of amino acids forming natural proteins. Examples of the substitution include those within a group of uncharged polar amino acids (Gly, Asn, Gln, Ser, Thr, Cys, Tyr), a group of branched-chain amino acids (Leu, Val, Ile), a group of neutral amino acids (Gly, Ile, Val, Leu, Ala, Met, Pro), a group of neutral amino acids containing a hydrophilic side chain (Asn, Gln, Thr, Ser, Tyr, Cys), a group of acidic amino acids (Asp, Glu), a group of basic amino acids (Arg, Lys, His), or a group of aromatic amino acids (Phe, Tyr, Trp). Amino acid substitutions within each of these groups are preferred since the properties of polypeptides are known to be less likely to change in such cases.

Incidentally, the LOX-1 protein may be a recombinant LOX-1 protein. The “recombinant LOX-1 protein” is a protein obtained by expressing a gene (LOX-1 gene) encoding LOX-1 protein obtained by the gene cloning technology, in a gene expression system using a host cell. Unless otherwise specified, the LOX-1 gene in present description means the human LOX-1 gene. The LOX-1 gene includes the wild type and mutant types. The wild-type human LOX-1 gene is a gene encoding human LOX-1 protein having the amino acid sequence represented by SEQ ID NO:2. Specific examples of the wild-type human LOX-1 gene include a polynucleotide having the base sequence represented by SEQ ID NO:1. The “mutant-type LOX-1 gene” means a polynucleotide consisting of a base sequence encoding the mutant-type LOX-1 protein. The recombinant LOX-1 protein may be prepared by expressing the LOX-1 gene in a host cell according to a conventional method in the art, or a commercially available recombinant LOX-1 protein may be used.

### (3) Part Thereof

The “part thereof” means a partial fragment of the LOX-1 protein, which is a region keeping the LAB-binding ability based on the receptor-ligand activity. Specific examples of the “part thereof” include the soluble form of LOX-1 protein.

The “soluble form of LOX-1 protein” (often referred to as “sLOX-1 protein” in the present description) means a peptide fragment consisting of the extracellular region of the LOX-1 protein. LOX-1 protein has a neck domain in the N-terminus of the extracellular domain. The neck domain presented in the N-terminus of the extracellular domain has a site highly sensitive to protease, and it is known that, when the protein is cleaved in this site, the extracellular region becomes a free state to be released to the outside of the cell, resulting in its appearance in blood. Since sLOX-1 protein has the binding region to LAB, it keeps the same LAB-binding ability as that of full-length LOX-1. When the sLOX-1 protein is a wild-type protein, examples of the sLOX-1 protein include: a polypeptide corresponding to position 61 to position 273 of the amino acid sequence of LOX-1 protein represented by SEQ ID NO:2, the polypeptide consisting of the 213 amino acids consisting of the amino acid sequence represented by SEQ ID NO:3; a polypeptide corresponding to position 91 to position 273 of the amino acid sequence of LOX-1 protein represented by SEQ ID NO:2, the polypeptide consisting of the 183 amino acids consisting of the amino acid sequence represented by SEQ ID NO:4; and a polypeptide corresponding to position 94 to position 273 of the amino acid sequence of LOX-1 protein represented by SEQ ID NO:2, the polypeptide consisting of the 180 amino acids consisting of the amino acid sequence represented by SEQ ID NO:5. When the LOX-1 protein is a peptide fragment consisting of the extracellular region of a mutant-type LOX-1 protein, examples of the LOX-1 protein include: polypeptides derived from the amino acid sequence as any represented by SEQ ID Nos:3 to 5 with deletions, substitutions or additions of one or more amino acids; and polypeptides having 90% or more, 91% or more, 92% or more, 93% or more, 94% or more, 95% or more, 96% or more, 97% or more, 98% or more, or 99% or more to the amino acid sequence represented by any of SEQ ID NOs:3 to 5.

The above-described part (of LOX-1 protein) may be part of a recombinant LOX-1 protein. Such part of the recombinant LOX-1 protein can be obtained by, for example, expressing a LOX-1 gene fragment encoding a desired region in the LOX-1 protein by a gene expression system.

The LAB-capturing device included in the kit for determining KD of the present invention has a configuration immobilizing LOX-1 protein on the base material surface. Thus, the LOX-1 protein used for the LAB-capturing device is more preferably the partial fragment of LOX-1 protein, such as sLOX-1 protein, which is in a free state and which keeps the LAB-binding ability, rather than full-length LOX-1 immobilized on a biomembrane through the transmembrane domain.

### 1-3-2. LAB-Detecting Agent

The “LAB-detecting agent” means an agent with a specific binding ability to LAB. The LAB-detecting agent may be consisting of a peptide, nucleic acid, or low molecular weight compound, or a combination thereof.

### (1) Peptide

When the LAB-detecting agent is consisting of a peptide, specific examples of the agent include, but are not limited to, antibodies and active fragments thereof, peptide aptamers, and LAB receptor proteins.

### (i) Antibodies and Active Fragments Thereof

The antibody that may be used as the LAB-detecting agent is an anti-LAB antibody capable of immunologically and specifically binding to LAB as an antigen; or a fragment of the antibody with LAB-binding ability.

The species from which the antibody is derived is not limited. The antibody may be derived from an animal including mammals and birds. Examples of the animal include mice, rats, guinea pigs, rabbits, goats, donkeys, sheep, camels, horses, chickens, and humans.

The type of the antibody can be used any of a polyclonal antibody, monoclonal antibody, recombinant antibody, and a synthesized antibody, and a combination thereof.

The “polyclonal antibody” means a group of a plurality of kinds of immunoglobulins capable of recognizing, and binding to, different epitopes of the same antigen. The polyclonal antibody can be obtained by immunizing an animal with a target molecule (LAB, in present description) as an antigen, followed by collection of the antibody from serum of the animal. A polyclonal antibody obtained by using LAB as the antigen is referred to as “anti-LAB polyclonal antibody” in present description.

The “monoclonal antibody” means a group of clones of a single immunoglobulin. Each immunoglobulin forming a monoclonal antibody comprises a common frame work region (hereinafter, referred to as “FR”) and a common complementarity determining region (hereinafter, referred to as “CDR”), and is capable of recognizing, and binding to, the same epitope of the same antigen. The monoclonal antibody can be obtained from hybridomas derived from a single cell. A monoclonal antibody obtained using LAB as the antigen is referred to as “anti-LAB monoclonal antibody” in present description.

A typical immunoglobulin molecule is a tetramer consisting of two pairs of two polypeptide chains called heavy chain and light chain, which are linked to each other through disulfide bonds. The heavy chain is consisting of a heavy-chain variable region (H chain V region; hereinafter, referred to as “VH”) located in the N-terminus, and a heavy-chain constant region (H chain C region; hereinafter, referred to as “CH”) located in the C-terminus. The light chain is consisting of a light-chain variable region (L chain V region; hereinafter, referred to as “VL”) located in the N-terminus, and a light-chain constant region (L chain C region; hereinafter, referred to as “CL”) located in the C-terminus. Among these, VH and VL are especially important because they are involved in the binding specificity of the antibody. Each of VH and VL is consisting of about 110 amino acid residues, and comprises three CDRs (CDR1, CDR2, and CDR3) directly associated with the binding specificity to the antigen, and four FRs (FR1, FR2, FR3, and FR4) that function as a skeletal structure of the variable region, in the order of FR1, CDR1, FR2, CDR2, FR3, CDR3, and FR4 from the N-terminus. The CDRs are known to form a three-dimensional structure complementary to the antigen molecule, to determine the specificity of the antibody (E. A. Kabat et al. 1991. Sequences of proteins of immunological interest, Vol. 1, eds. 5, NIH publication). In the variable region, the CDRs and the FRs are arranged in the order of FR1, CDR1, FR2, CDR2, FR3, CDR3, and FR4 in the direction from the N-terminus to the C-terminus. In the immunoglobulin molecule, VL pairs with VH each other to form a dimer, to thereby form an antigen-binding site.

When the antibody is a polyclonal antibody or a monoclonal antibody, IgG, IgM, IgA, IgE, and IgD are known as classes of the immunoglobulin molecule. The class of the antibody of the present invention may be any of these classes. The class is preferably IgG.

Regarding the specific method for the preparation of the hybridoma producing the monoclonal antibody that recognizes, and binds to, LAB or a peptide fragment thereof, an antibody preparation method conventionally known in the art may be employed.

The “recombinant antibody” in present description includes a chimeric antibody, a humanized antibody, and a multi-specific antibody. The “chimeric antibody” means an antibody prepared by combination of amino acid sequences of antibodies derived from different animals, wherein the V region of an antibody is replaced with the V region of another antibody. Examples of the chimeric antibody include an antibody replaced the V region of an anti-human LAB mouse monoclonal antibody that specifically binds to human LAB, with the V region of a human antibody such that the V region is derived from the mouse and the C region is derived from the human. Examples of the “humanized antibody” include a graft antibody replaced the CDRs (CDR1, CDR2, and CDR3) in the V region of an anti-human LAB monoclonal antibody which is derived from a non-human mammal such as a mouse and which specifically binds to human LAB, with the CDRs of a human monoclonal antibody. The “multi-specific antibody” means a multivalent antibody, that is, an antibody comprising plural antigen-binding sites in a single molecule, wherein the antigen-binding sites bind to different epitopes. In cases of an antibody comprising two antigen-binding sites such as IgG, examples of the multi-specific antibody include a bispecific antibody whose antigen-binding sites bind to the same or different LABs described in the first aspect.

The “synthetic antibody” in present description means an antibody synthesized by using a chemical method or a recombinant DNA method. Examples of the synthetic antibody include monomeric polypeptide molecules prepared by linking one or more VLs and one or more VHs of a specific antibody/antibodies through a linker peptide(s) having an appropriate length(s) and sequence(s), and multimeric polypeptides thereof. Specific examples of such polypeptides include single chain fragments of variable region (scFv) (see Pierce Catalog and Handbook, 1994-1995, Pierce Chemical Co., Rockford, Ill.), diabodies, triabodies, and tetrabodies. In an immunoglobulin molecule, VL and VH are usually located in separate polypeptide chains (L chain and H chain). “Single-chain Fv” is a synthetic antibody fragment having a structure in which the V regions on these two polypeptide chains are linked to each other through a flexible linker having a sufficient length to include them in a single polypeptide chain. In a single-chain Fv, both V regions are capable of self-assembling to form a single functional antigen-binding site. The single-chain Fv can be obtained by incorporating a recombinant DNA encoding it into a phage genome using a known technique, and then allowing its expression. “Diabody” is a molecule based on a single-chain Fv dimer structure, and has two functional antigen-binding sites (Holliger et al., 1993, Proc. Natl. Acad. Sci. USA 90: 6444-6448). For example, when the length of the linker is less than 12 amino acid residues, the two variable regions in the single-chain Fv is structurally incapable of self-assembling. However, by forming a diabody and allowing the two single-chain Fvs to interact with each other, VL of one Fv chain can be allowed to assemble with VH of the other Fv chain, and two functional antigen-binding sites can be formed as a result (Marvin et al., 2005, Acta Pharmacol. Sin. 26:649-658). Further, by adding a cysteine residue to the C-terminus of each single-chain Fv, disulfide bond between the two Fv chains can be formed, so that a stable diabody can be formed (Olafsen et al., 2004, Prot. Engr. Des. Sel. 17:21-27). Although the diabody is a divalent antibody fragment as described above, its antigen-binding sites do not need to bind to the same epitope, and may recognize and specifically bind to different epitopes. The diabody may thus have dual specificity. Similarly to the diabody, “triabody” and “tetrabody” have trimer and tetramer structures based on single-chain Fv structures. They are trivalent and tetravalent antibody fragments, respectively, and may be multi-specific antibodies.

In present description, the “active fragment thereof” means a partial fragment of the polyclonal antibody or monoclonal antibody, which fragment is a polypeptide chain having an activity substantially equivalent to the antigen-specific binding activity of the antibody, or a complex of the polypeptide chain. Examples of the fragment include an antibody portion including at least one antigen-binding site, that is, a polypeptide chain comprising at least a pair of VL and VH, or a complex thereof. Specific examples of the fragment include a large number of sufficiently characterized antibody fragments produced by cleaving immunoglobulin with various peptidases. More specific examples of the fragment include Fab, F(ab′)2, and Fab′. Fab is a fragment produced by cleavage of an IgG molecule with papain at a position in the N-terminus relative to the disulfide bonds of the hinge region. Fab is consisting of: a polypeptide consisting of VH, and of CH1, which is the domain adjacent to VH among the three domains forming CH (CH1, CH2, and CH3); and a light chain. F(ab′)2 is a dimer of Fab′ produced by cleavage of an IgG molecule by pepsin at a position in the C-terminus relative to the disulfide bonds of the hinge region. Since Fab′ includes the hinge region, it has a slightly longer H chain compared to Fab. However, it has substantially the same structure as Fab (Fundamental Immunology, Paul ed., 3d ed., 1993). Fab′ can be obtained by reducing F(ab′)2 under mild conditions, and then cleaving the disulfide linkage in the hinge region. All of these antibody fragments include an antigen-binding site, and are capable of specifically binding to a target molecule corresponding to an antigen.

### (ii) Peptide Aptamer

“Aptamer” is a ligand molecule having an ability to strongly and specifically bind to a target material based on the three-dimensional structure. Depending on the type of the molecule forming each aptamer, aptamers can be roughly divided into nucleic acid aptamers and peptide aptamers.

“Peptide aptamer” means an aptamer consisting of amino acids, and is a peptide molecule of 1 to 6 kD capable of recognizing a surface structure of a target molecule to specifically bind to the target material based on the three-dimensional structure, similar to antibodies. The target molecule of the peptide aptamer in present description is LAB. The peptide aptamer can be produced using the phage display method or the cell surface display method. Regarding the production method for the peptide aptamer, the peptide aptamer may be prepared according to a method known in the art. For example, one may refer to Whaley, S.R., et al., 2000, Nature, 405, 665-668.

(iii) LAB Receptor Protein

Examples of the “LAB receptor protein” include the above-described LOX-1 protein or the fragment thereof having LAB-binding ability. Here, description of the LOX-1 or the like is omitted since it has already been described in detail.

### (2) Nucleic Acid

When the LAB-detecting agent is consisting of nucleic acid, specific examples of the agent include, but are not limited to, nucleic acid aptamers.

### (i) Nucleic Acid Aptamer

Among the aptamers described above, “nucleic acid aptamer” means an aptamer consisting of nucleic acid. The nucleic acid forming the nucleic acid aptamer may be any of DNA, RNA, and combinations thereof. The nucleic acid aptamer may contain a chemically modified nucleic acid such as PNA, LNA/BNA, methylphosphonate-modified DNA, phosphorothioate-modified DNA, or 2′-O-methyl-modified RNA, if necessary.

The nucleic acid aptamer can be prepared by a method known in the art using LAB as a target molecule. For example, in cases of an RNA aptamer, it can be prepared by in vitro selection using the SELEX (systematic evolution of ligands by exponential enrichment) method. The SELEX method is a method in which RNA molecules bound to LAB are selected and collected from an RNA pool consisting of a large number of RNA molecules each having a random sequence region and primer-binding regions at both ends thereof, and then the collected RNA molecules are amplified by RT-PCR reaction, followed by performing transcription using the obtained cDNA molecules as templates, to provide the resulting transcripts as the RNA pool for the subsequent round. This process is repeated for several to several ten rounds of cycles, to select RNAs having stronger binding capacities to LAB. The lengths of the base sequences of the random sequence region and the primer-binding regions are not limited. In general, the random sequence region has a length within the range of 20 to 80 bases, and each primer-binding region has a length within the range of 15 to 40 bases. An RNA molecule finally obtained by the above method is used as the LAB-binding RNA aptamer. The SELEX method is a known method. More specifically, the method may be carried out according to, for example, Pan et al. (Proc. Natl. Acad. Sci. 1995, U.S.A.92: 11509-11513).

Each of the LAB-detecting agents described above may be labeled, if necessary. As the label, a labeling material known in the art can be used. In cases of an antibody or a peptide aptamer, it may be labeled with, for example, a fluorescent dye (fluorescein, FITC, rhodamine, Texas Red, Cy3, or Cy5), a fluorescent protein (such as PE, APC, or GFP), an enzyme (such as horseradish peroxidase, alkaline phosphatase, or glucose oxidase), a radioisotope (such as ³H, or ³⁵S), or biotin or (strept)avidin. In cases of a nucleic acid aptamer, examples of the labeling material include radioisotopes (such as ³²P, ³H, and ¹⁴C), DIG, biotin, fluorescent dyes (such as FITC, Texas, cy3, cy5, cy7, FAM, HEX, VIC, JOE, Rox, TET, Bodipy 493, NBD, and TAMRA), and luminescent substances (such as acridinium esters). The label may be two or more different labels. The LAB-detecting agent labeled with the labeling material can be a useful tool for detection of LAB.

### 2. Method for Determining Kawasaki Disease (Method for Determining KD)

### 2-1. Summary

A second aspect of the present invention is a method for determining KD. The present invention is constituted such that LAB as the marker for determining KD described in the first aspect contained in a blood sample of a subject is detected using, as a capturing material(s), LOX-1 protein and/or the part thereof, and such that KD is determined based on the amount of LAB. The method for determining KD of the present invention enables direct and objective determination of KD in a subject suspected of having KD, which had so far been inevitably dependent on clinical findings and exclusion diagnosis.

### 2-2. Method

The method for determining KD of the present invention comprises a measurement step and a determination step. Each step is specifically described below.

### 2-2-1. Measurement Step

The “measurement step” is a step for quantifying LAB contained per unit amount of blood sample collected from a subject suspected of having KD, wherein a measured value is obtained by measuring the amount of LAB.

The “subject” in present description means an animal individual to be subjected to the method for determining KD of the present invention. Examples of the animal individual to be subjected to the method for determining KD of the present invention include mammals such as human, dog, cat, horse, cow, sheep, goat, camel, rabbit, ferret, hamster, and mouse. The animal individual is preferably a human. The subject is preferably an individual suspected of having KD.

The “subject suspected of having KD” in present description means a subject exhibiting symptoms that are found in KD patients, based on clinical findings and/or the like. It is, in principle, an individual suspected of having KD according to diagnosis by a doctor or the like. In present description, the subject means especially a KD patient in the acute phase, subacute phase, or chronic phase. Patients in the convalescent phase, who are not exhibiting the symptoms, are regarded as patients recovered from KD or patients who had previously had KD, and distinguished from KD patients. The diagnosis is made mainly by combination of an interview, a clinical course, findings in physical examination, muscle pathological findings, and the like.

The “healthy individual” in present description means a non-KD individual who is obviously at least free of KD, and means, in principle, an individual belonging to the same species as the subject, which individual has been diagnosed as being free of KD by a doctor or the like. The healthy individual is preferably an individual having no disease.

The “healthy-individual group” in present description means a group consisting of plural healthy individuals of the same species. The number of individuals is not limited as long as it is two or more. The number of individuals is preferably 5 or more, more preferably 10 or more, still more preferably 15 or more. Each individual constituting this population is preferably an individual of the same species and the same sex as the subject, and its physical conditions such as the age, body height, and body weight are preferably the same as or similar to those of the subject.

The “measured value in a healthy-individual group” is a measured value obtained by measuring the amount of the marker for determining KD, that is, the amount of LAB, contained per unit amount of blood sample collected from each individual constituting the healthy-individual group. This measured value is, in principle, a measured value obtained in the measurement step using the same type of blood sample as in, and using the same measurement method as, the method by which the measured value of the subject having KD was obtained. In the healthy-individual group, the amount of each marker for determining KD in each sample may be preliminarily measured by each measurement method, and the resulting measured value may be used to prepare a database for providing the measured value in this group.

The “blood sample” in present description corresponds to whole blood, serum, plasma, or interstitial fluid.

In present description, the “collected blood sample” means a blood sample collected from each of the subject and the prior-described healthy-individual group. The method for the collection is not limited, and may be a known blood collection method. For example, peripheral blood may be collected from a peripheral vein or the like using a syringe. The blood sample may be used by the determining method immediately after the collection. Alternatively, the blood sample may be cooled on ice after the collection, and then subjected to centrifugation to obtain serum or plasma, followed by storing it in a deep freezer, and thawing it for use if necessary. Before the present step or during the present step, if necessary, the blood sample may be concentrated, or may be diluted with physiological saline or the like, or an anticoagulant such as heparin may be added to the blood sample.

The “unit amount” is a predetermined unit in terms of the volume or weight, and examples of the unit amount include microliter (μL), milliliter (mL), microgram (μg), milligram (mg), and gram (g).

In the present description, the “measured value” is a value indicating the amount of LAB measured in the present step. This amount may be a relative amount in terms of the fluorescence intensity, luminescence intensity, turbidity, absorbance, amount of radiation, ionic strength, or concentration, or may be an absolute amount such as the weight or volume of LAB contained in a sample.

In the present step, the amount of LAB, which is a marker for determining KD, contained in a blood sample derived from a subject is measured.

The amount of the blood sample required for use in the method for determining KD of the present invention may be at least 100 μL, preferably 200 μL when whole blood is used. When serum or plasma is used, the amount may be at least 50 μL, preferably 100 μL.

### (1) Measurement Method

LAB is a lipoprotein. Therefore, as the measurement method, a known lipoprotein quantification method can be used, and the method is not limited. Examples of the method include an immunological detection method, a receptor-ligand binding analysis method, an aptamer analysis method, gel filtration HPLC, mass spectrometry, and combinations thereof.

### (i) Immunological Detection Method

The “immunological detection method” is a most common method of the measurement, wherein a target molecule is used as an antigen, and an antibody specifically binding thereto or a fragment thereof is used to allow formation of an immune complex with the target molecule, followed by detection and quantification of the target molecule. Since LAB corresponds to the target molecule in the present invention, the immunological detection method means a method for measuring the amount of LAB contained in a blood sample using an anti-LAB antibody or a fragment thereof.

Examples of the immunological detection method include enzyme immunoassay, fluorescence immunoassay, luminescence immunoassay, the surface plasmon resonance method (SPR method), the quartz crystal microbalance (QCM) method, radioimmunoassay (RIA), turbidimetric immunoassay, latex agglutination immunoassay, the latex turbidimetric method, the particle agglutination reaction method, the gold colloid method, capillary electrophoresis, western blotting, and immunohistochemistry (the immunostaining method). All these methods are known methods, and may be carried out, in principle, according to ordinary methods in the art. For example, one may refer to methods described in Current protocols in Protein Sciences, 1995, John Wiley & Sons Inc.; Current protocols in Immunology, 2001, John Wiley & Sons Inc.; Green &Sambrook, Molecular Cloning, 2012, Fourth Ed., Cold Spring Harbor Laboratory Press Cold Spring Harbor, New York; Japan Society of Clinical Pathology (ed.), “The Official journal of Japanese Society of Laboratory Medicine, Extra Edition vol. 53, Immunoassay for Clinical Examination: Techniques and Application”, Rinsho-byori publication association, 1983; Eiji Ishikawa et al. (eds.), “Enzyme Immunoassay”, 3rd edition, Igaku-Shoin Ltd. 1987; Tsunehiro Kitagawa et al. (eds.), “Protein, Nucleic Acid and Enzyme, Extra Edition No. 31, Enzyme Immunoassay”, Kyoritsu Shuppan Co., Ltd., 1987; Minoru Irie (ed.), “Radioimmunoassay”, Kodansha Scientific, Ltd., 1974; Minoru Irie (ed.), “Radioimmunoassay 2”, Kodansha Scientific, Ltd., 1979; Kazuhiro Nagara and Hiroshi Handa (eds.), Experimental Methods of Real-Time Analysis of Interactions of Biological Substances. Springer-Verlag (Tokyo), 1988; Toyosaka Moriizumi and Takamichi Nakamoto, Sensor Engineering, Shokodo Co., Ltd., 1997; and the like.

“Enzyme immunoassay” is a method in which a primary antibody bound to a target molecule is detected through a labeled secondary antibody or the like, and the color optical density or fluorescence intensity based on the label is used to quantify the target molecule. Examples of the enzyme immunoassay include a method in which an anti-LAB antibody as a primary antibody bound to LAB is captured with a labeled secondary antibody that binds to the primary antibody, and then the LAB is indirectly measured based on the signal intensity or the like from the label. The ELISA method and the sandwich ELISA method are also included in this method.

The “surface plasmon resonance (SPR) method” is a method in which a material adsorbing to a surface of a metallic thin film is highly sensitively detected and quantified utilizing the surface plasmon resonance phenomenon, which is the phenomenon that, when a laser beam is radiated to a metallic thin film at various angles, remarkable attenuation of reflected light occurs at a specific incidence angle (resonance angle). In the present invention, for example, LOX-1 protein or an anti-LAB antibody is immobilized on a metallic thin film surface, and the surface moiety of the metallic thin film is subjected to blocking treatment, followed by allowing a blood sample to flow through the metallic thin film surface, to detect and quantify LAB based on the difference between the measured values obtained before and after the flow of the sample. The detection and quantification by the surface plasmon resonance method may be carried out using, for example, an SPR sensor commercially available from Biacore Inc.

The “quartz crystal microbalance (QCM) method” is a mass measurement method utilizing the phenomenon that adsorption of a material on the surface of an electrode attached to a quartz crystal causes a decrease in the resonance frequency of the quartz crystal depending on the mass of the material, wherein a very small amount of adsorbing material is quantitatively analyzed based on the amount of change in the resonance frequency. The detection and quantification of the target molecule by this method may be carried out using, for example, a commercially available QCM sensor, similarly to the case of the SPR method. In the present invention, LAB can be quantified by, for example, antigen-antibody reaction between LOX-1 protein or an anti-LAB antibody immobilized on an electrode surface, and LAB in a sample.

### (ii) Receptor-Ligand Binding Analysis Method

The “receptor-ligand binding analysis method” is a method applicable when the target molecule is a ligand or receptor. The method utilizes the receptor-ligand activity to capture one of these present invention in a sample using the other, to measure the amount of the former. Since LAB, which is the target molecule in the present invention, is a ligand molecule, and since its specific receptor is LOX-1 protein, this method is also applicable. Specific examples of the method include modified methods of enzyme immunoassay, fluorescence immunoassay, luminescence immunoassay, radioimmunoassay (RIA), the surface plasmon resonance method (SPR method), the quartz crystal microbalance (QCM) method, turbidimetric immunoassay, latex agglutination immunoassay, the latex turbidimetric method, the particle agglutination reaction method, the gold colloid method, or the like used in immunological detection methods. For example, LOX-1 protein or a fragment thereof having LAB-binding ability may be immobilized on a base material, and the protein complex (receptor-ligand complex) formed by binding to LAB in the blood sample may be measured. In cases of enzyme immunoassay, indirect measurement is possible by a modified sandwich ELISA method in which the LAB-LOX-1 protein complex on the base material is detected with a labeled anti-LAB antibody. In cases of the SPR method or the QCM method, direct measurement of the LAB-LOX-1 protein complex formed on a metallic thin film surface or an electrode surface is possible.

(iii) Aptamer Analysis Method

The “aptamer analysis method” is a method in which a nucleic acid aptamer or a peptide aptamer is used to quantify a target molecule. The method is basically the same as the immunological detection method except that an aptamer that specifically binds to the target molecule is used instead of the antigen-binding antibody. In the present invention, a LAB-binding aptamer (LAB-binding RNA aptamer, LAB-binding DNA aptamer, or LAB-binding peptide aptamer) may be used in the same manner as the anti-LAB antibody in the immunological detection method, to detect and measure LAB in blood.

### (iv) Mass Spectrometry

“Mass spectrometry” is a method in which a sample is ionized under high vacuum, and then the resulting ions are electromagnetically separated to analyze materials in the sample. When a predetermined target molecule in a sample is to be detected, detection and quantification of the target molecule during viewing are possible by comparison of a mass spectrum obtained using the target molecule as an authentic sample and a mass spectrum of the sample. In the present invention, LAB corresponds to the target molecule.

Examples of the “mass spectrometry” include high-performance liquid chromatography (LC-MS), high-performance liquid chromatograph-tandem mass spectrometry (LC-MS/MS), gas chromatography-mass spectrometry (GC-MS), gas chromatography-tandem mass spectrometry (GC-MS/MS), capillary electrophoresis-mass spectrometry (CE-MS), and ICP-mass spectrometry (ICP-MS).

In the present step, for correction of the measured value of the subject and the measured value in the healthy-individual group, a known protein expected to exhibit no quantitative difference in the unit amount among samples may be used as an endogenous control. Examples of such an endogenous control include albumin.

### 2-2-2. Determination Step

The “determination step” is a step of determining whether the subject suffers from KD based on a measured value of the subject obtained by the measurement step.

The term “based on a measured value of the subject” means “depending on the value of a measured value of the subject” obtained as a result of the measurement step. More specifically, it means, for example, determination of KD based on a cut-off value, or based on a statistically significant difference between a measured value of the subject and a measured value in a healthy-individual group.

### (i) Determination Method Based on Cut-Off Value

The “determination method based on a cut-off value” is a method in which the measured value of the subject is compared with a predetermined cut-off value to determine whether the subject suffers from KD based on the result of the comparison.

In present description, the “cut-off value” means a boundary value for classifying measured values into positive values and negative values. The positive values herein indicate that the subject is likely to have KD, and the negative values indicate that the subject is unlikely to have KD. The method for setting of the cut-off value is not limited, and the value may be set according to a method known in the field of statistics. For example, in a measured-value group consisting of measured values of KD patients and healthy individuals, a specifying percentile may be used as the cut-off value. For example, when almost all measured values of KD patients are included in the values higher than the measured value corresponding to 90 percentile in the measured-value group, the measured value corresponding to 90 percentile is used as the cut-off value. In this case, when the measured value of a subject is higher than the cut-off value, the subject may be determined to be positive, in other words, the possibility that the subject has KD may be determined to be high. On the other hand, when the measured value of the subject is not more than the cut-off value, the subject may be determined to be negative, in other words, the possibility that the subject has KD may be determined to be low.

### (ii) Determination Method Based on Statistically Significant Difference

In the determination method based on a statistically significant difference, whether the subject suffers from KD is determined based on whether or not the measured value of the subject is statistically significantly higher than the measured value of the healthy-individual group.

In present description, examples of the “statistical significance” include cases where the significance level (level of significance) of the obtained value is low, such as cases of p<0.05 (less than 5%), p<0.01 (less than 1%), or p<0.001 (less than 0.1%). Here, the “p (value)” means a probability that a hypothesis becomes true by chance in a hypothesized distribution of a statistic in a statistical test. Thus, the lower the p value, the closer the hypothesis to the truth. The “statistically significant difference” means that there is a significant difference between a measured value of a subject and a measured value of a population when their difference is statistically treated. The test method in the statistical treatment is not limited, and a known test method capable of determining whether the subject suffers from significance may be appropriately used. For example, Student's t-test can be used.

When KD is determined based on a statistically significant difference in present description, if the measured value of the marker for determining KD is significantly higher in the subject than in the healthy-individual group, the possibility that the subject has KD is determined to be high. On the other hand, if there is no significant difference in the measured value of the marker for determining KD between the subject and the healthy-individual group, the possibility that the subject does not have KD is determined to be high.

### 3. Marker for Determining Presence of Kawasaki Disease (Marker for Determining Presence of KD)

### 3-1. Summary

A third step of the present invention is a marker for determining KD. Regarding the marker for determining KD of the present invention, LAB is used as a biomarker for determining KD. By measuring the amount of the marker contained in a blood sample of a subject using the method for determining KD described in the second aspect, whether the subject suffers from KD can be determined.

### 3-2. Configuration

The marker for determining KD is consisting of LAB or part thereof keeping LOX-1-binding ability.

As described above, LAB is an apolipoprotein that is also called oxidized LDL (modified LDL), and contains apoprotein B (apoB) as a protein component. Unless otherwise specified, the apoB in present description is human apoB. apoB includes the wild type and mutant types. More specifically, the wild-type apoB is a polypeptide having the amino acid sequence represented by SEQ ID NO:6. The mutant-type apoB in present description means a polypeptide which is the same as the wild-type apoB except for a mutation(s) that has/have occurred in part thereof, which polypeptide keeps the binding ability to LOX-1. Examples of the mutant-type apoB include, but are not limited to: polypeptides derived from the amino acid sequence represented by SEQ ID NO:6 by deletions, substitutions or additions of one or more amino acids; and polypeptides having 90% or more, 91% or more, 92% or more, 93% or more, 94% or more, 95% or more, 96% or more, 97% or more, 98% or more, or 99% or more to the amino acid sequence represented by SEQ ID NO:6.

The region and the amino acid length of the part of LAB are not limited as long as it has the binding ability to LOX-1.

### EXAMPLES

### (Object)

Using LOX-1 protein, which is a LAB receptor, it is verified that the amount of oxidized LDL (LAB) present in plasma is significantly increased in patients with KD compared to those in healthy individuals.

### (Methods)

### (1) Sample Preparation

### (Preparation of Blood Samples)

Blood was collected from 16 KD patients diagnosed with KD according to a KD diagnosis guideline (Ayusawa, M., et al., 2005, Pediatr Int 47: 232-234), after obtaining informed consent. As the controls, blood was similarly collected from five normal control individuals and seven disease control individuals. Here, the “normal control individuals” are control individuals who have a history of food allergy, but who are uninfected and have no fever. The “disease control individuals” are patients with a febrile disease such as pneumonia, gastroenteritis, bacterial infection, or viral infection (human metapneumovirus or RS virus).

The blood collection from each patient was performed in the acute phase before the application of intravenous immunoglobulin (IVIG) therapy. Eight patients were subjected to IVIG therapy, and, one month after disappearance of the symptoms, the same amount of blood was collected therefrom to obtain follow-up samples.

For obtaining plasma, 1 to 1.5 mL of blood was collected into a CBC spitz tube (EDTA2Na), and then immediately centrifuged to collect the supernatant. The collected plasma was stored at -30° C. until use.

### (Preparation of Recombinant Soluble LOX-1 Protein Solution)

LOX-1 protein to be used for capturing LBA in plasma based on the receptor-ligand activity was prepared. The LOX-1 protein used in the present Example was prepared by diluting a human-derived recombinant soluble form of LOX-1 (sLOX-1) protein consisting of the amino acid sequence represented by SEQ ID NO:3 (Yokohama Bio Research and Supply, Inc.) with PBS(-) to a final concentration of 5 μL/mL. The recombinant sLOX-1 protein corresponds to position 61 to position 273 of the amino acid sequence represented by SEQ ID NO:3, and keeps the receptor-ligand activity to LAB.

### (Preparation of Blocking Solution)

Each of Block Ace Powder (KAC Co., Ltd.) and sucrose was dissolved in distilled water, to prepare 4% Block Ace solution and 30% sucrose solution. On the day before the preparation of an sLOX-1-immobilized plate, a blocking solution (3% Block Ace, 2% sucrose) was prepared at a ratio of 4% Block Ace: distilled water: 30% sucrose =9.0 mL: 2.2 mL: 0.8 mL (=basic ratio).

### (Preparation of Antibody Solution)

An HRP-labeled chicken anti-human apolipoprotein monoclonal antibody (HUC20: Creative Biolabs) was dissolved in PBS, to prepare an HRP (Horse Radish Peroxidase)-labeled anti-apolipoprotein B antibody solution (HRP-HUC20 antibody solution). HRP-HUC20 antibody specifically recognizes the extracellular domain of human apolipoprotein B. It is labeled with HRP.

(2) Preparation of sLOX-1-Immobilized Plate

To each well of a 96-well microplate (PerkinElmer Japan Co., Ltd.), 100 μL of the recombinant sLOX-1 protein solution was dispensed, and the solution was stirred at 1000 rpm for 3 minutes using a plate shaker (IKA (registered trademark) Japan K.K.). Thereafter, the plate was sealed, and left to stand at 4° C. for not less than 16 hours. After the time has passed, the seal was peeled off, and the well was washed once with 380 μL of a washing liquid (Takara Bio Inc.). After removing the washing liquid, 300 μL of a blocking solution was dispensed into the well. The plate was then sealed again, and left to stand at 4° C. for 18 to 24 hours. After the time has passed, the seal was peeled off, and the blocking solution was removed by suction, followed by drying the plate in a clean bench at room temperature (25 to 26° C.) for 18 to 24 hours. The dried plate was provided as an sLOX-1-immobilized plate.

### (3) Measurement of Plasma Level of LAB by Enzyme Immunoassay

(Binding of sLOX-1 to LAB)

Each well of the sLOX-1-immobilized plate prepared in (2) was washed three times with 380 μL of a washing liquid (Takara Bio Inc.) before use, and then water was sufficiently removed therefrom. Subsequently, 100 _{N}L of a plasma sample (derived from a KD patient in the acute phase, derived from a patient in the convalescent phase who had previously had KD, derived from a normal control individual, or derived from a disease control individual) was dispensed into each well. The plate was then sealed, and incubated at room temperature for 2 hours. After removing the plasma sample, the plate was washed three times with 380 μL of a washing liquid (Takara Bio Inc.), and then water was sufficiently removed therefrom.

### (Detection and Quantification of LAB)

While the HRP-labeled HUC20 antibody solution prepared in (1) was diluted with a diluent (0.4% Block Ace/PBS) to finally achieve 420-fold dilution, 100 μL of the dilution was dispensed into each well. The plate was then sealed, and stirred at 1000 rpm for 1 minute, followed by incubation at room temperature for 1 hour. Subsequently, the antibody solution was removed, and each well was washed three times with 380 μL of a washing liquid (Takara Bio Inc.), followed by sufficiently removing water therefrom.

For detection of the HRP-labeled HUC20 antibody bound to LAB, a luminescence liquid was prepared by mixing Peroxide Solution and Luminol/Enhancer Solution included in SuperSignal™ ELISA Pico Chemiluminescent Substrate (Thermo Fisher Scientific Inc.) at a ratio of 1:1, and 100 μL of the luminescence liquid was dispensed into each well. After stirring the mixture using a plate shaker at 1000 rpm for 1 minute, luminescence emitted due to the HRP activity was detected using a plate reader (Infinite (registered trademark) 200 PRO: Tecan Japan Co., Ltd.), and quantification was carried out based on the luminescence intensity.

### (Results)

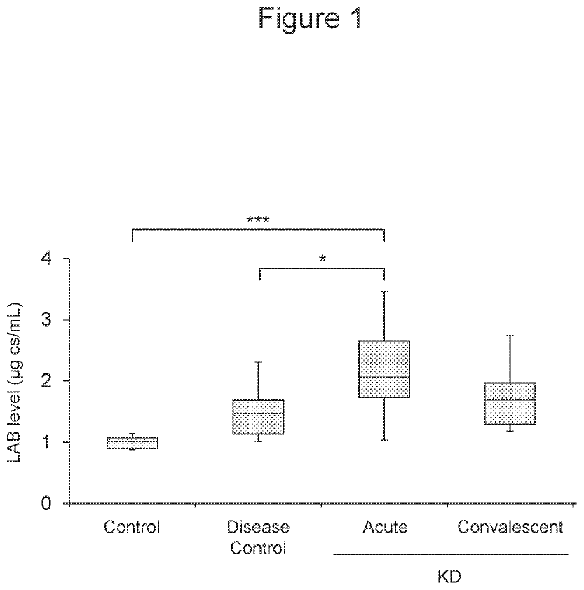

FIG. 1 shows the results. As illustrated in this figure, the LAB level in the acute phase (Acute) was significantly higher than those of the normal control (Control) and the disease control (Disease Control). On the other hand, the LAB level in the convalescent phase (Convalescent) showed no significance. As a result of comparison between the values of the LAB patients in the acute phase and the normal control using ROC, the cut-off value was found to be 1.55.

From these results, it was found that the amount of LAB is significantly increased in blood of KD patients in the acute phase, and that the amount decreases after recovery from KD by application of IVIG therapy. These results thus suggest that LAB in blood may potentially be a biomarker for determination of KD.

It was also found that sLOX-1 protein can be used as a LAB-capturing material for detection of LAB in blood.

All publications, patents, and patent applications cited in the present description are hereby incorporated as they are by the citation.

1. A kit for determining Kawasaki disease, comprising a capturing device for Lox-1 ligand containing apolipoprotein B (LAB),
wherein the LAB-capturing device comprises lectin-like oxidized low-density lipoprotein receptor 1 protein (LOX-1 protein) immobilized on a surface of a base material and/or part thereof having LAB-binding ability.

2. The kit according to claim 1, wherein the LOX-1 protein is selected from the group consisting of:
(a) a polypeptide consisting of the amino acid sequence represented by SEQ ID NO:2;
(b) a polypeptide derived from the amino acid sequence represented by SEQ ID NO:2 by deletions, substitutions or additions of one or more amino acids; and
(c) a polypeptide having 90% or more an amino acid identity to the amino acid sequence represented by SEQ ID NO:2.

3. The kit according to claim 1, wherein the part is selected from the group consisting of:
(d) a polypeptide having the amino acid sequence represented by any of SEQ ID NOs:3 to 5;
(e) a polypeptide derived from the amino acid sequence represented by any of SEQ ID NOs:3 to 5 by deletions, substitutions or additions of one or more amino acids; and
(f) a polypeptide having 90% or more an amino acid identity to the amino acid sequence represented by any of SEQ ID NOs:3 to 5.

4. The kit according to claim 1, further comprising a LAB-detecting agent.

5. The kit according to claim 4, wherein the LAB-detecting agent is labeled.

6. The kit according to claim 4, wherein the LAB-detecting agent is an anti-LAB antibody or a fragment thereof having LAB-binding ability.

7. A method for determining Kawasaki disease, comprising:
a measurement step of measuring the amount of LAB contained per unit amount of blood sample collected from a subject to obtain a measured value of the amount of LAB; and
a determination step of determining whether the subject suffers from Kawasaki disease based on the measured value obtained in the measurement step.

8. The method according to claim 7, wherein, in the determination step, the subject is determined to have Kawasaki disease when the measured value obtained in the measurement step is higher than a predetermined cut-off value, or when the measured value obtained in the measurement step is significantly higher than the amount of LAB contained per unit amount of blood sample collected from a healthy-individual group.

9. The method for according to claim 7, wherein the measurement in the measurement step is carried out using receptor-ligand activity between LAB, and LOX-1 protein and/or part thereof having LAB-binding ability.

10. The method use according to claim 7, wherein the blood sample is any of blood, serum, and plasma.

11. (canceled)


SEQUENCE LISTING (6 sequences)

SEQ ID NO: 1 - DNA, 822 bp, Homo sapiens
atgacttttgatgacctaaagatccagactgtgaaggaccagcctgatgagaagtcaaatggaaaaaaagctaaaggtcttcagtttctttactctccatggtggtgcctggctgctgcgactctaggggtcctttgcctgggattagtagtgaccattatggtgctgggcatgcaattatcccaggtgtctgacctcctaacacaagagcaagcaaacctaactcaccagaaaaagaaactggagggacagatctcagcccggcaacaagcagaagaagcttcacaggagtcagaaaacgaactcaaggaaatgatagaaacccttgctcggaagctgaatgagaaatccaaagagcaaatggaacttcaccaccagaatctgaatctccaagaaacactgaagagagtagcaaattgttcagctccttgtccgcaagactggatctggcatggagaaaactgttacctattttcctcgggctcatttaactgggaaaagagccaagagaagtgcttgtctttggatgccaagttgctgaaaattaatagcacagctgatctggacttcatccagcaagcaatttcctattccagttttccattctggatggggctgtctcggaggaaccccagctacccatggctctgggaggacggttctcctttgatgccccacttatttagagtccgaggcgctgtctcccagacatacccttcaggtacctgtgcatatatacaacgaggagctgtttatgcggaaaactgcattttagctgccttcagtatatgtcagaagaaggcaaacctaagagcacagtga

SEQ ID NO: 2 - protein, 273 aa, Homo sapiens
MTFDDLKIQTVKDQPDEKSNGKKAKGLQFLYSPWWCLAAATLGVLCLGLVVTIMVLGMQLSQVSDLLTQEQANLTHQKKKLEGQISARQQAEEASQESENELKEMIETLARKLNEKSKEQMELHHQNLNLQETLKRVANCSAPCPQDWIWHGENCYLFSSGSFNWEKSQEKCLSLDAKLLKINSTADLDFIQQAISYSSFPFWMGLSRRNPSYPWLWEDGSPLMPHLFRVRGAVSQTYPSGTCAYIQRGAVYAENCILAAFSICQKKANLRAQ

SEQ ID NO: 3 - protein, 213 aa, Homo sapiens
SQVSDLLTQEQANLTHQKKKLEGQISARQQAEEASQESENELKEMIETLARKLNEKSKEQMELHHQNLNLQETLKRVANCSAPCPQDWIWHGENCYLFSSGSFNWEKSQEKCLSLDAKLLKINSTADLDFIQQAISYSSFPFWMGLSRRNPSYPWLWEDGSPLMPHLFRVRGAVSQTYPSGTCAYIQRGAVYAENCILAAFSICQKKANLRAQ

SEQ ID NO: 4 - protein, 183 aa, Homo sapiens
AEEASQESENELKEMIETLARKLNEKSKEQMELHHQNLNLQETLKRVANCSAPCPQDWIWHGENCYLFSSGSFNWEKSQEKCLSLDAKLLKINSTADLDFIQQAISYSSFPFWMGLSRRNPSYPWLWEDGSPLMPHLFRVRGAVSQTYPSGTCAYIQRGAVYAENCILAAFSICQKKANLRAQ

SEQ ID NO: 5 - protein, 180 aa, Homo sapiens
ASQESENELKEMIETLARKLNEKSKEQMELHHQNLNLQETLKRVANCSAPCPQDWIWHGENCYLFSSGSFNWEKSQEKCLSLDAKLLKINSTADLDFIQQAISYSSFPFWMGLSRRNPSYPWLWEDGSPLMPHLFRVRGAVSQTYPSGTCAYIQRGAVYAENCILAAFSICQKKANLRAQ

SEQ ID NO: 6 - protein, 4563 aa, Homo sapiens
MDPPRPALLALLALPALLLLLLAGARAEEEMLENVSLVCPKDATRFKHLRKYTYNYEAESSSGVPGTADSRSATRINCKVELEVPQLCSFILKTSQCTLKEVYGFNPEGKALLKKTKNSEEFAAAMSRYELKLAIPEGKQVFLYPEKDEPTYILNIKRGIISALLVPPETEEAKQVLFLDTVYGNCSTHFTVKTRKGNVATEISTERDLGQCDRFKPIRTGISPLALIKGMTRPLSTLISSSQSCQYTLDAKRKHVAEAICKEQHLFLPFSYKNKYGMVAQVTQTLKLEDTPKINSRFFGEGTKKMGLAFESTKSTSPPKQAEAVLKTLQELKKLTISEQNIQRANLFNKLVTELRGLSDEAVTSLLPQLIEVSSPITLQALVQCGQPQCSTHILQWLKRVHANPLLIDVVTYLVALIPEPSAQQLREIFNMARDQRSRATLYALSHAVNNYHKTNPTGTQELLDIANYLMEQIQDDCTGDEDYTYLILRVIGNMGQTMEQLTPELKSSILKCVQSTKPSLMIQKAAIQALRKMEPKDKDQEVLLQTFLDDASPGDKRLAAYLMLMRSPSQADINKIVQILPWEQNEQVKNFVASHIANILNSEELDIQDLKKLVKEALKESQLPTVMDFRKFSRNYQLYKSVSLPSLDPASAKIEGNLIFDPNNYLPKESMLKTTLTAFGFASADLIEIGLEGKGFEPTLEALFGKQGFFPDSVNKALYWVNGQVPDGVSKVLVDHFGYTKDDKHEQDMVNGIMLSVEKLIKDLKSKEVPEARAYLRILGEELGFASLHDLQLLGKLLLMGARTLQGIPQMIGEVIRKGSKNDFFLHYIFMENAFELPTGAGLQLQISSSGVIAPGAKAGVKLEVANMQAELVAKPSVSVEFVTNMGIIIPDFARSGVQMNTNFFHESGLEAHVALKAGKLKFIIPSPKRPVKLLSGGNTLHLVSTTKTEVIPPLIENRQSWSVCKQVFPGLNYCTSGAYSNASSTDSASYYPLTGDTRLELELRPTGEIEQYSVSATYELQREDRALVDTLKFVTQAEGAKQTEATMTFKYNRQSMTLSSEVQIPDFDVDLGTILRVNDESTEGKTSYRLTLDIQNKKITEVALMGHLSCDTKEERKIKGVISIPRLQAEARSEILAHWSPAKLLLQMDSSATAYGSTVSKRVAWHYDEEKIEFEWNTGTNVDTKKMTSNFPVDLSDYPKSLHMYANRLLDHRVPQTDMTFRHVGSKLIVAMSSWLQKASGSLPYTQTLQDHLNSLKEFNLQNMGLPDFHIPENLFLKSDGRVKYTLNKNSLKIEIPLPFGGKSSRDLKMLETVRTPALHFKSVGFHLPSREFQVPTFTIPKLYQLQVPLLGVLDLSTNVYSNLYNWSASYSGGNTSTDHFSLRARYHMKADSVVDLLSYNVQGSGETTYDHKNTFTLSCDGSLRHKFLDSNIKFSHVEKLGNNPVSKGLLIFDASSSWGPQMSASVHLDSKKKQHLFVKEVKIDGQFRVSSFYAKGTYGLSCQRDPNTGRLNGESNLRFNSSYLQGTNQITGRYEDGTLSLTSTSDLQSGIIKNTASLKYENYELTLKSDTNGKYKNFATSNKMDMTFSKQNALLRSEYQADYESLRFFSLLSGSLNSHGLELNADILGTDKINSGAHKATLRIGQDGISTSATTNLKCSLLVLENELNAELGLSGASMKLTTNGRFREHNAKFSLDGKAALTELSLGSAYQAMILGVDSKNIFNFKVSQEGLKLSNDMMGSYAEMKFDHTNSLNIAGLSLDFSSKLDNIYSSDKFYKQTVNLQLQPYSLVTTLNSDLKYNALDLTNNGKLRLEPLKLHVAGNLKGAYQNNEIKHIYAISSAALSASYKADTVAKVQGVEFSHRLNTDIAGLASAIDMSTNYNSDSLHFSNVFRSVMAPFTMTIDAHTNGNGKLALWGEHTGQLYSKFLLKAEPLAFTFSHDYKGSTSHHLVSRKSISAALEHKVSALLTPAEQTGTWKLKTQFNNNEYSQDLDAYNTKDKIGVELTGRTLADLTLLDSPIKVPLLLSEPINIIDALEMRDAVEKPQEFTIVAFVKYDKNQDVHSINLPFFETLQEYFERNRQTIIVVLENVQRNLKHINIDQFVRKYRAALGKLPQQANDYLNSFNWERQVSHAKEKLTALTKKYRITENDIQIALDDAKINFNEKLSQLQTYMIQFDQYIKDSYDLHDLKIAIANIIDEIIEKLKSLDEHYHIRVNLVKTIHDLHLFIENIDFNKSGSSTASWIQNVDTKYQIRIQIQEKLQQLKRHIQNIDIQHLAGKLKQHIEAIDVRVLLDQLGTTISFERINDVLEHVKHFVINLIGDFEVAEKINAFRAKVHELIERYEVDQQIQVLMDKLVELAHQYKLKETIQKLSNVLQQVKIKDYFEKLVGFIDDAVKKLNELSFKTFIEDVNKFLDMLIKKLKSFDYHQFVDETNDKIREVTQRLNGEIQALELPQKAEALKLFLEETKATVAVYLESLQDTKITLIINWLQEALSSASLAHMKAKFRETLEDTRDRMYQMDIQQELQRYLSLVGQVYSTLVTYISDWWTLAAKNLTDFAEQYSIQDWAKRMKALVEQGFTVPEIKTILGTMPAFEVSLQALQKATFQTPDFIVPLTDLRIPSVQINFKDLKNIKIPSRFSTPEFTILNTFHIPSFTIDFVEMKVKIIRTIDQMLNSELQWPVPDIYLRDLKVEDIPLARITLPDFRLPEIAIPEFIIPTLNLNDFQVPDLHIPEFQLPHISHTIEVPTFGKLYSILKIQSPLFTLDANADIGNGTTSANEAGIAASITAKGESKLEVLNFDFQANAQLSNPKINPLALKESVKFSSKYLRTEHGSEMLFFGNAIEGKSNTVASLHTEKNTLELSNGVIVKINNQLTLDSNTKYFHKLNIPKLDFSSQADLRNEIKTLLKAGHIAWTSSGKGSWKWACPRFSDEGTHESQISFTIEGPLTSFGLSNKINSKHLRVNQNLVYESGSLNFSKLEIQSQVDSQHVGHSVLTAKGMALFGEGKAEFTGRHDAHLNGKVIGTLKNSLFFSAQPFEITASTNNEGNLKVRFPLRLTGKIDFLNNYALFLSPSAQQASWQVSARFNQYKYNQNFSAGNNENIMEAHVGINGEANLDFLNIPLTIPEMRLPYTIITTPPLKDFSLWEKTGLKEFLKTTKQSFDLSVKAQYKKNKHRHSITNPLAVLCEFISQSIKSFDRHFEKNRNNALDFVTKSYNETKIKFDKYKAEKSHDELPRTFQIPGYTVPVVNVEVSPFTIEMSAFGYVFPKAVSMPSFSILGSDVRVPSYTLILPSLELPVLHVPRNLKLSLPDFKELCTISHIFIPAMGNITYDFSFKSSVITLNTNAELFNQSDIVAHLLSSSSSVIDALQYKLEGTTRLTRKRGLKLATALSLSNKFVEGSHNSTVSLTTKNMEVSVATTTKAQIPILRMNFKQELNGNTKSKPTVSSSMEFKYDFNSSMLYSTAKGAVDHKLSLESLTSYFSIESSTKGDVKGSVLSREYSGTIASEANTYLNSKSTRSSVKLQGTSKIDDIWNLEVKENFAGEATLQRIYSLWEHSTKNHLQLEGLFFTNGEHTSKATLELSPWQMSALVQVHASQPSSFHDFPDLGQEVALNANTKNQKIRWKNEVRIHSGSFQSQVELSNDQEKAHLDIAGSLEGHLRFLKNIILPVYDKSLWDFLKLDVTTSIGRRQHLRVSTAFVYTKNPNGYSFSIPVKVLADKFIIPGLKLNDLNSVLVMPTFHVPFTDLQVPSCKLDFREIQIYKKLRTSSFALNLPTLPEVKFPEVDVLTKYSQPEDSLIPFFEITVPESQLTVSQFTLPKSVSDGIAALDLNAVANKIADFELPTIIVPEQTIEIPSIKFSVPAGIVIPSFQALTARFEVDSPVYNATWSASLKNKADYVETVLDSTCSSTVQFLEYELNVLGTHKIEDGTLASKTKGTFAHRDFSAEYEEDGKYEGLQEWEGKAHLNIKSPAFTDLHLRYQKDKKGISTSAASPAVGTVGMDMDEDDDFSKWNFYYSPQSSPDKKLTIFKTELRVRESDEETQIKVNWEEEAASGLLTSLKDNVPKATGVLYDYVNKYHWEHTGLTLREVSSKLRRNLQNNAEWVYQGAIRQIDDIDVRFQKAASGTTGTYQEWKDKAQNLYQELLTQEGQASFQGLKDNVFDGLVRVTQEFHMKVKHLIDSLIDFLNFPRFQFPGKPGIYTREELCTMFIREVGTVLSQVYSKVHNGSEILFSYFQDLVITLPFELRKHKLIDVISMYRELLKDLSKEAQEVFKAIQSLKTTEVLRNLQDLLQFIFQLIEDNIKQLKEMKFTYLINYIQDEINTIFSDYIPYVFKLLKENLCLNLHKFNEFIQNELQEASQELQQIHQYIMALREEYFDPSIVGWTVKYYELEEKIVSLIKNLLVALKDFHSEYIVSASNFTSQLSSQVEQFLHRNIQEYLSILTDPDGKGKEKIAELSATAQEIIKSQAIATKKIISDYHQQFRYKLQDFSDQLSDYYEKFIAESKRLIDLSIQNYHTFLIYITELLKKLQSTTVMNPYMKLAPGELTIIL